(19) United States
(12) Patent Application Publication
(10) Pub. No.: US 2019/0117437 A1
(43) Pub. Date: Apr. 25, 2019

(54) EXTERNAL FIXATION DEVICE FOR ORTHOPEDICS OF FOREARM AND PALM

(71) Applicant: Xieping ZHU, Shanghai (CN)

(72) Inventor: Xieping ZHU, Shanghai (CN)

(21) Appl. No.: 15/790,042

(22) Filed: Oct. 22, 2017

(51) Int. Cl. A61F 5/058; A61F 13/04

(52) U.S. Cl. CPC A61F 5/05858; A61F 2013/00578; A61F 13/048; A61F 5/05866

(57) ABSTRACT

An external fixation device for fractured forearms and/or hands. The device includes an arm-shaped main body and a cavity. The main body includes: a forepart which corresponds to a hand of a patient, and a rear part which corresponds to a segment extending from the hand to an elbow joint of the patient. The main body includes a first edge and a second edge. The first edge of the main body is provided with a flexible cotton member, and a placket is connected to the outer side of the flexible cotton member. The second edge of the main body is provided with a plurality of elastic belts. The rear part of the main body includes an opening which is located at a position corresponding to the elbow joint of the patient and communicates with the filler cavity.

### BACKGROUND OF THE INVENTION

### Field of the Invention

The invention relates to the field of fixation of bones, and, in particular, to an external fixation device for fractured forearms and/or hands of patients suffering from bone fractures.

### Description of the Related Art

Existing materials and methods for fixation of fractured bones include fixation by plaster high polymer bandages, prefabricated models, and internal supports.

Fixation by plaster and fixation by high polymer bandages are not suitable for treatment of complicated bone fractures, and the X-rays cannot penetrate the plaster and bandages to examine bone healing. When fixation is performed at maximum tissue swelling post-fracture, after the edema subsides, a gap results between the external fixation device and the broken bone, destabilizing the reduced bone. When the swelling of the tissue is developing post-fracture and the fixation by plaster or bandages is conducted, the plaster or bandages may restrict further tissue swelling, which may lead to tissue damage and necrosis, and adversely affect the treatment.

Fixation by prefabricated models requires frequent hospital visits. This is time consuming and may be difficult for patients.

Fixation by internal supports is risky as it may cause new fractures and infections.

### SUMMARY OF THE INVENTION

In view of the above-described problems, it is one objective of the invention to provide an external fixation device for fractured forearms and/or hands. The external fixation device is adapted to treat the bone fractures of emergency patient in conformity to the dynamic change of swelling of injured parts and peripherals from the early stage to the late stage.

To achieve the above objective, in accordance with one embodiment of the invention, there is provided an external fixation device for fractured forearms and/or hands, the device comprising an arm-shaped main body and a cavity, the main body comprising: a forepart which corresponds to a hand of a patient, and a rear part which corresponds to a segment from the hand to an elbow joint of the patient; the main body comprising a first edge and a second edge, the first edge and the second edge approaches to each other to form the cavity for accommodating the hand and arm of the patient.

The main body comprises an inner layer, an outer layer, and a filler cavity sandwiched between the inner layer and the outer layer; the inner layer and the outer layer are made of elastic cotton materials; the first edge of the main body is provided with a flexible cotton member, and a placket is connected to an outer side of the flexible cotton member; the second edge of the main body is provided with a plurality of elastic belts; the rear part of the main body comprises an opening which is located at a position corresponding to the elbow joint of the patient and communicates with the filler cavity; and a hook and loop fastener is disposed to surround the opening; the placket of the first edge and the plurality of elastic belts of the second edge are connected via a connecting piece.

The binding force of the flexible cotton member with regard to a body surface of the patient is between 1.4 g/mm² and 5 g/mm².

The method of fixation for fractured forearms and/or hands using the device, the method comprises: filling polyurethane slurry into the filler cavity via the opening of the rear part of the main body, allowing the polyurethane slurry to solidify, drawing close the first edge and the second edge of the main body in 360° through the connecting piece to wrap broken forearms and/or hands of a patient, allowing the polyurethane slurry to further solidify to yield a rigid fastener, and locking the flexible cotton member, the placket, and the connecting piece.

In a class of this embodiment, an arc angle of the rigid fastener after fixing the forearms and/or hands of the patient is between 200 degrees and 240 degrees.

In a class of this embodiment, the flexible cotton member comprises an inner core of polyurethane.

In a class of this embodiment, the connecting piece comprises a hook-type piece sewed on the placket and a loop-type piece sewed on the plurality of elastic belts.

In a class of this embodiment, the placket is made of two layers of elastic cotton materials.

The external device for fixation of bones of forearms and/or hands of the invention has appropriate tensile force and strength, and the X-rays can penetrate the device, so that the recovery status of the fractured parts can be examined clearly. Meanwhile, because it is convenient for the fixation device to ventilate and clean, the medical personnel can freely disassemble the external device according to the requirements of patients so as to clean and treat the skin of injured parts, prevent skin inflammation and festering. Compared to the fixation by internal supports and other traditional methods adopted in recent years, the fixation device of the invention is better in terms of treatment time and comprehensive cost performance Compared to traditional tube-shaped fixation by plaster and high polymer bandages and high polymer bandage supports, the device realizes the dynamic fixation of swollen pats of patients and close-fitting fixation.

### BRIEF DESCRIPTION OF THE DRAWINGS

FIG. 1 is a structural diagram of an external device for fixation of bones of forearms and/or hands in an unfolded state of the disclosure; and

FIG. 2 is a structural diagram of an external device for fixation of bones of forearms and/or hands in a folded state of the invention.

### DETAILED DESCRIPTION OF THE EMBODIMENTS

The invention is described in detail below with reference to the accompanying drawings and embodiments.

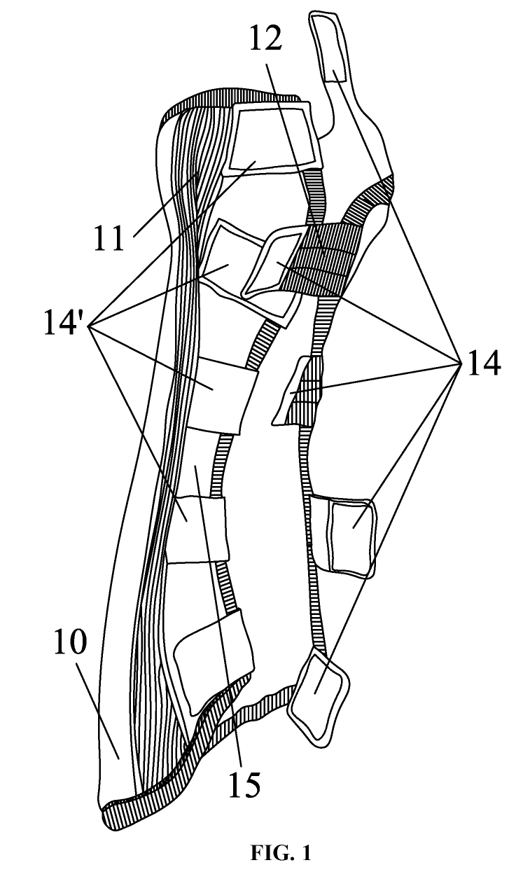

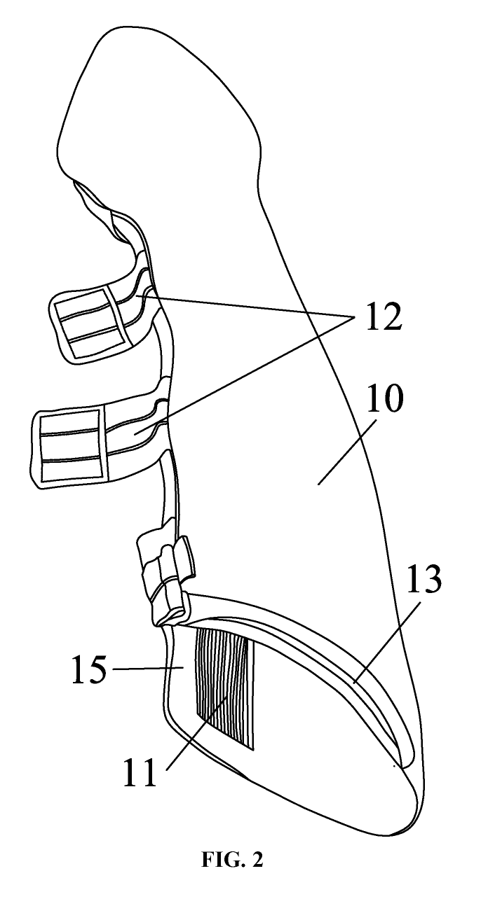

Please refer to FIGS. 1 and 2, an external fixation device for fractured forearms and/or hands of the invention comprises an arm-shaped main body and a cavity, the main body comprising: a forepart which corresponds to a hand of a patient, and a rear part which corresponds to a segment from the hand to an elbow joint of the patient.

The main body comprises an inner layer, an outer layer, and a filler cavity 10 sandwiched between the inner layer and the outer layer. The first edge of the main body is provided with a flexible cotton member 11, and a placket 15 is connected to an outer side of the flexible cotton member 11. The second edge of the main body is provided with a plurality of elastic belts 12.

The inner layer and the outer layer are made of elastic cotton materials; the rear part of the main body comprises an opening which is located at a position corresponding to the elbow joint of the patient and communicates with the filler cavity; and a hook and loop fastener 13 is disposed to surround the opening.

The flexible cotton member 11 is made of flexible cotton materials which adapt to shape according to human bodies. The flexible cotton member 11 comprises an inner core of polyurethane. The elastic force of the flexible cotton member 11 is corresponding to the external tension force generated when the hand or forearm is swollen, that is, the binding force of the flexible cotton member 11 with regard to a body surface of the patient is between 1.4 g/mm² and 5 g/mm². The flexible cotton member 11 is friendly to the skin and makes the skin breathable.

The placket 15 is made of two layers of elastic cotton materials, which are the same as that of the two layers of the main body.

A plurality of elastic belts 12 are sewed on the second edge of the main body 10 at intervals; a preferred number of the elastic belts 12 is five and the elastic belt 12 arranged on the forefront of the main body 10 passes the parts between thumbs and index fingers. The placket 15 of the first edge and the plurality of elastic belts 12 of the second edge are connected via a connecting piece. The connecting piece comprises a hook-type piece 14 sewed on the placket and a loop-type piece 14′ sewed on the plurality of elastic belts 12.

The main body 10 is filled with polyurethane paste (disclosed in the U.S. Pat. No. 9,617,370) through the opening the opening of the rear part of the main body 10; the polyurethane paste is evened by rolling on an operating platform; after the polyurethane paste enters the solid-phase stage (softening), the arm-shaped main body wraps the forearm and/or hand of a human body according to the shapes of the forearm and/or hand in a way of 360-degree enclosure through the connecting piece. Therefore, the main body 10 forms a rigid fixation part, and the flexible cotton member 11, the placket 15, the elastic belts 12, and the connecting piece are locked. The arc angle of the rigid fastener after fixing the forearms and/or hands of the patient is between 200 degrees and 240 degrees.

When the shapes of the fractured limbs of the patients constantly change in the presence of edema, the external fixation device of the invention also alters accordingly to match the fractured parts of the patients. That is, the fixation is dynamic. In short, when the limbs swell, the external fixation device of the invention always keeps dynamic change so as to closely fit the human bodies; during the recovery of edema parts, the external fixation device of the invention correspondingly shrinks until the edema parts become normal.

While the external fixation device of the invention fixes injured parts of fractured patients in emergency, one side of the main body 10 is provided with the flexible cotton member 11 and the placket 15 successively; the other side of the main body 10 is provided with the elastic belt 12; the placket 15 of the arm-shaped main body and the elastic belt 12 are connected via the connecting piece; when the arm-shaped main body wraps the forearms and/or hands in 360-degree enclosure through the connecting piece, the dynamic, firm, and close fixation is achieved. The elastic force of the flexible cotton member 11 on one side of the main body 10 corresponds to the outward force produced by the swelling of the injured parts, realizing the dynamic fixation on the basis of real-time memories. The external fixation device of the invention comprises a rigid fixation part and an elastic fixation part, and is totally different from elastic bandages in clinical use.

The external device for fixation of bones of forearms and/or hands of the invention has appropriate tensile force and strength, and the X-rays can penetrate the device, so that the recovery status of the fractured parts can be examined clearly. Meanwhile, because it is convenient for the fixation device to ventilate and clean, the medical personnel can freely disassemble the external device according to the requirements of patients so as to clean and treat the skin of injured parts, prevent skin inflammation and festering. Compared to the fixation by internal supports and other traditional methods adopted in recent years, the fixation device of the invention is better in terms of treatment time and comprehensive cost performance Compared to traditional tube-shaped fixation by plaster and high polymer bandages and high polymer bandage supports, the device realizes the dynamic fixation of swollen pats of patients and close-fitting fixation.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. An external fixation device for fractured forearms and/or hands, the device comprising an arm-shaped main body and a cavity, the main body comprising: a forepart which corresponds to a hand of a patient, and a rear part which corresponds to a segment from the hand to an elbow joint of the patient; the main body comprising a first edge and a second edge, the first edge and the second edge approaches to each other to form the cavity for accommodating the hand and arm of the patient;
wherein:
the main body comprises an inner layer, an outer layer, and a filler cavity sandwiched between the inner layer and the outer layer;
the inner layer and the outer layer are made of elastic cotton materials;
the first edge of the main body is provided with a flexible cotton member, and a placket is connected to an outer side of the flexible cotton member;
the second edge of the main body is provided with a plurality of elastic belts;
the rear part of the main body comprises an opening which is located at a position corresponding to the elbow joint of the patient and communicates with the filler cavity; and a hook and loop fastener is disposed to surround the opening;
the placket of the first edge and the plurality of elastic belts of the second edge are connected via a connecting piece; and
a binding force of the flexible cotton member with regard to a body surface of the patient is between 1.4 g/mm² and 5 g/mm².

2. The device of claim 1, wherein the flexible cotton member comprises an inner core of polyurethane.

3. The device of claim 1, wherein the connecting piece comprises a hook-type piece sewed on the placket and a loop-type piece sewed on the plurality of elastic belts.

4. The device of claim 1, wherein the placket is made of two layers of elastic cotton materials.

5. The device of claim 3, wherein the placket is made of two layers of elastic cotton materials.

6. A method of fixation for fractured forearms and/or hands using the device of claim 1, the method comprising:
filling polyurethane slurry into the filler cavity via the opening of the rear part of the main body;
allowing the polyurethane slurry to solidify, drawing close the first edge and the second edge of the main body in 360° through the connecting piece to wrap broken forearms and/or hands of a patient;
allowing the polyurethane slurry to further solidify to yield a rigid fastener; and
locking the flexible cotton member, the placket, and the connecting piece.

7. The method of claim 6, wherein an arc angle of the rigid fastener after fixing the forearms and/or hands of the patient is between 200 degrees and 240 degrees.
